(19) United States
(12) Patent Application Publication
(10) Pub. No.: US 2008/0051784 A1
(43) Pub. Date: Feb. 28, 2008

(54) Bone repositioning apparatus and methodology

(76) Inventor: Sohrab Gollogly, Carmel by the Sea, CA (US)

Correspondence Address: ROBERT D. VARITZ, P.C., 4915 SE 33RD PLACE, PORTLAND, OR, 97202, US

(21) Appl. No.: 11/498,618

(22) Filed: Aug. 3, 2006

(51) Int. Cl. A61F 2/30

(52) U.S. Cl. 606/61

(57) ABSTRACT

A method and apparatus for producing a change in the spacing between a pair of selected, spaced skeletal bones in a patient utilizing a single-surgery, elongate selectively adjustable, length-change bone-spacing device The device includes (a) an elongate, pre-stressed, length-change, spring-force component having a relaxed condition toward which it is self-biased, and in which relaxed condition it is either one of longer or shorter than when it is in its pre-stressed condition, (b) a pair of spaced, relatively moveable bone drivers coupleable to selected bones, and drivingly connected, one each, to opposite ends of the spring-force component, and (c) a two-state, RF-activated, stress-control constraining medium embedding at least a portion of the length of the spring-force component, and having a hardened state wherein it immobilizes that length portion against any length change, and a softened, fluid state to which it converts reversibly under the influence of received radio-frequency (RF) energy of a defined character which is specific to the constraining medium, and in which softened state, the constraining medium permits length change of the spring-force component's embedded length portion.

### BACKGROUND OF THE INVENTION

This invention relates generally to the field of medicine, and in particular to a branch of medicine which is concerned with the often companion subjects of thoracic insufficiency syndrome and scoliosis—deformity conditions which involves distortions in relative positioning and in shaping, especially in young children, regarding the ribs and the spine, respectively.

For many years there has been an intense interest in addressing these two conditions, and over those years, a number of specific practices and associated implantable corrective devices have been employed, typically involving multiple surgeries, to introduce positional/dispositional correcting forces between different bones to urge them toward relative positional and dispositional normalcy.

By way of useful background information, there are several publications, mentioned hereinbelow, which set the stage for a clear understanding of the problems presented by the above-mentioned deformity conditions. These background documents include: “The Characteristics of Thoracic Insufficiency Syndrome Associated with Fused Ribs and Congenital Scoliosis”, The Journal of Bone & Joint Surgery, Volume 85-A, Number 3, March 2003, pages 399-408, inclusive; “The Effect of Opening Wedge Thoracostomy on Thoracic Insufficiency Syndrome Associated with Fused Ribs and Congenital Scoliosis”, The Journal of Bone & Joint Surgery, Volume 86-A, Number 8, August 2004, pages 1659-1674, inclusive,; U.S. Pat. No. 5,720,746, Soubeiran, Feb. 24, 1998; and U.S. Pat. No. 6,796,984 B2, Souberian, Sep. 28, 2004; and U.S. Pat. No. 6,918,910 B2, Jul. 19, 2005.

The two just-listed technical articles provide good background disclosures regarding the two problems which are specifically addressed by the present invention. They also discuss certain well-known prior art approaches to remedying these problems.

The '746 and '984 patents describe different, prior-art, radio-frequency (RF) controlled, spring-biased, length-changing displacement devices which may be implanted in a patient to produce desired spacing changes between different pairs of bones. In these devices there is included a phrase-change “constraining” material, or medium, which normally has a hardened state, but which softens under the influence of exposure to a selected RF field of energy. Softening of this medium creates fluidizing of a small region of localized interengagement which exists between (a) this region of the medium, and (b) at least one of a pair of relatively moveable bone-drivers, thus to enable shifting of the bone drivers' relative positions translationally under the influence of a biasing spring, thereby producing a space-changing driving force between a pair of bones which are effectively connected, one each, to the two bone drivers. The spring provided in these prior art devices which exerts a biasing force on the two bone drivers is not itself engaged with the constraining medium.

In each of these two patents, and as was just generally indicated above, there is disclosed a structure in which there is a pair of spring-based, relatively moveable bone drivers, at least one of which includes a small projecting portion that locally engages the mentioned RF-softenable phase-change constraining material. With respect to the prior art reflected in these two patents, it is significant to note that the spring-biasing component per se is not directly engaged with, and is therefore not directly controlled by, the RF-softenable material. This being the case, constraint imposed by the RF-softenable material against the making of a length-change adjustment, at least until that softenable material is exposed to selected RF radiation, is developed (1) through a condition involving interengagement between the bone-connected moveable drivers and the RF softenable material, rather than (2) through another kind of condition involving interengagement between the spring-biasing component per se and the RF-softenable material.

The significance of this last-made statement, in the context of the offerings of the present invention, is that it describes two, appreciably different, length-change operating conditions, (1) and (2), the first-mentioned one of which (the one described in the patents) offers less precision control over length-change adjustments than does the other, second-mentioned condition, a unique condition wherein constraint against length change is based, differently, upon direct interengagement between a spring-biasing component and a mass of RF softenable constraining material. This other condition is the one which is specifically employed as a new and special feature in embodiments disclosed herein of the present invention. More will be said about this unique, and improved, “interengagement” condition (condition 2) later in this specification.

Finally, the '910 patent discloses an implantable distraction device utilizing, for periodic adjustments that urge spacing changes between bones, periodic pressure-fluid actuation as a medium for producing bone-position-adjusting length-change in an implanted device. This implanted device, which thus does permit periodic adjustment once it has been implanted, nonetheless requires, effectively, a plurality of “surgery-like” procedures, or at least invasive procedures, in the sense that plural skin penetrations are required over time to inject actuating pressure-fluid to perform length- and spacing-change adjustments.

As will be seen from the detailed invention description which follows below, when that is read in conjunction with the accompanying drawings, the present invention offers a number of important improvements over the known prior art relating to the practice, and to the apparatus employed for this practice, of addressing the thoracic insufficiency syndrome and scoliosis issues described above.

### SUMMARY OF THE INVENTION

The present invention, as will now be explained, addresses the issue of producing a spacing change between selected bones in the context of using unique apparatus, and an associated unique methodology. More specifically, the invention offers an approach for changing bone spacing via a methodology which is, essentially, single-surgery, single-anatomical-invasion only, and which uses RF energy, of one or more selected characteristics (frequencies, etc.), to activate a spring-biased length-change device which is implanted inside a patient's anatomy. This length-change device, in a way which is only distantly related to the disclosures of the above-referred-to '746 and '984 patents, utilizes RF energy to soften, or relax in a melting fashion, from a hardened state, an appropriate RF-responsive constraining material (medium) so as to unconstrain, in a very precision-controlled way, a pre-stressed biasing spring. When such unconstraining occurs, the biasing spring is then permitted to act movingly on a pair of bone-connected drivers to shift associated connected bones either to open up or to close the space between these bones, depending upon whether the pre-stressed condition of the biasing spring is one of compression or one of tension.

Very specifically, in the approach proposed by the present invention, there is a direct, interactive connection and association—an embedding connection and association—between the RF-responsive constraining material mentioned and the spring biasing component per se, whereas, in the prior art illustrated in the '746 and '984 patents, the relevant operative interengagement is between an RF-softenable constraining medium and a small projection structure provided on one or both of the illustrated bone drivers. The embedded-style biasing-spring/RF-softenable medium interaction which characterizes the present invention distinguishes the approach of the invention from that which is proposed in the just-mentioned two U.S. patents. This distinction is important in that it promotes and enables very high-precision length-change adjustments, as will become apparent front the detailed description of the invention presented below.

Another feature of the invention involves the manner in which it may selectively be connected to bones through spacing-change bone drivers. This “manner” is one which permits a certain amount of otherwise normal translational relative motion (i.e., spacing-change motion), notwithstanding the existence of connections to bones established at opposite ends of the proposed length-change apparatus.

These and other features of the invention will now become more fully apparent as the detailed description thereof which follows below is read in conjunction with the accompanying drawings.

### DESCRIPTION OF THE DRAWINGS

FIG. 1 is a fragmentary, high-level, schematic illustration showing one form of bone-spacing apparatus, a distraction apparatus, made in accordance with the present invention, positioned for acting between a pair of next-adjacent ribs for the purpose of enlarging the spacing between those ribs to deal with a thoracic insufficiency syndrome condition.

FIG. 2 is like FIG. 1, except that it shows practice of the invention utilizing contraction apparatus to diminish the spacing between a pair of next-adjacent ribs.

FIG. 3 is another schematic, high-level, fragmentary illustration of similar bone-spacing apparatus interposed two portions of a single rib from which a part of that rib has been removed. The bone-spacing apparatus appearing in this figure is shown positioned, effectively, to extend the length of the illustrated rib.

FIG. 4 is yet another fragmentary, high-level, schematic view illustrating an implanted installation in a patient of three different bone-spacing apparatuses, two of which act between different pairs of next-adjacent ribs, and the third one of which is deployed to act between the upper rib pictured in FIG. 4 and the iliac buttress portion of a patient's pelvis. This figure illustrates both a thoracic insufficiency syndrome condition, and a scoliosis condition.

FIG. 5 is a high-level, schematic view further illustrating bone-spacing apparatus made in accordance with the invention, including an elongate coil spring which sits in a pre-stressed condition embedded within an RF-responsive constraining medium which is switchable between hardened and softened states in accordance with practice of the present invention. FIG. 5 is used herein to illustrate, alternatively, two different kinds of pre-stressed conditions for a biasing spring included in the apparatus of the invention, one of which conditions is a condition of compression, and the other of which is a condition of tension.

FIG. 6 is a high-level, schematic view of the apparatus of FIG. 5 picturing a positive length change based upon the assumption that the apparatus shown in FIG. 5 has its biasing spring pre-stressed in compression. Such a length change results from RF-softening of an RF-responsive constraining material which embeds a length region, or portion, of the mentioned under-compression biasing spring.

FIG. 7 illustrates, for the apparatus of FIG. 5, a negative length change which results from contraction of the biasing spring shown in FIG. 5 as a consequence of RF-softening of the RF-responsive constraining medium which embeds a length portion of that biasing spring. The assumption here, of course, is that the biasing spring pictured in FIG. 5 is in pre-stressed tension condition.

FIG. 8 is related to FIG. 6, and shows how a certain range of normal, relative, lateral translational motion between a pair of bones is permitted, notwithstanding the presence between those bones of a distraction-producing apparatus, like those shown in FIGS. 1 and 3.

FIG. 9 is like FIG. 8, except that it is related to FIG. 7, and shows another range of normal, relative, lateral, translational motion which is permitted between bones, also notwithstanding the presence between those bones of a contraction bone-spacing apparatus made in accordance with the invention.

FIG. 10 is a high-level, schematic illustration of a bone-spacing apparatus made in accordance with the present invention, with this figure being employed, as will be explained more fully hereinbelow, to visualize, alternatively, this apparatus being connected (a) between a pair of next-adjacent vertebral bodies, (b) between a vertebral body and a rib, and (c) between a vertebral body and the pelvis.

### DETAILED DESCRIPTION OF THE INVENTION

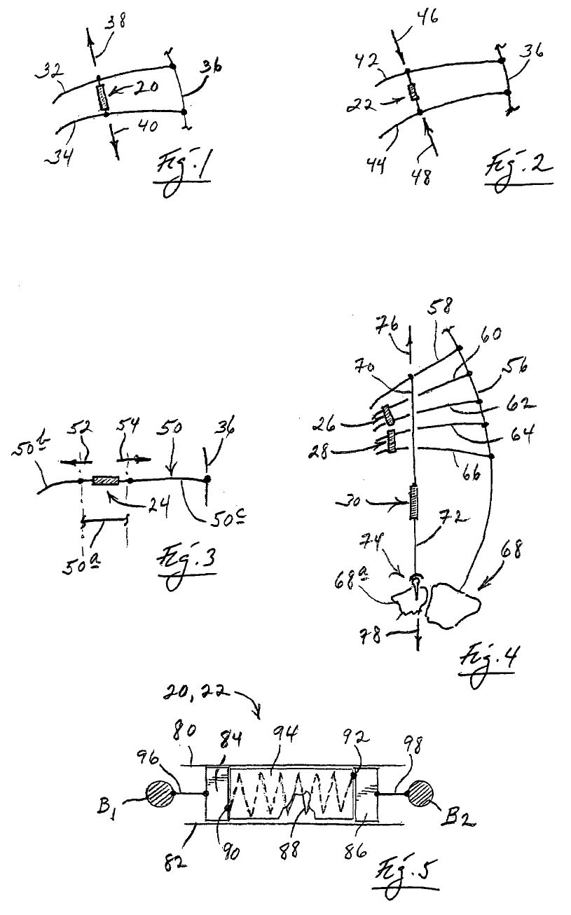

Turning now to the drawings, and referring first of all, somewhat sequentially, to FIGS. 1-5, inclusive, indicated generally at 20 in FIG. 1, at 22 in FIG. 2, at 24 in FIG. 3, and at 26, 28, 30 in FIG. 4 are similarly constructed bone-spacing devices, also referred to herein as bone-spacing apparatus. Each of these devices is also referred to herein (1) as a single-surgery, elongate, selectively adjustable length-change device, or apparatus, and also (2) as an elongate, unidirectional, distributed-RF-controllable, length-change force-applying instrumentality.

In FIG. 1, and as will be more fully explained shortly, opposite ends of device 20 are appropriately connected to a pair of spaced apart, next-adjacent ribs, or bones, 32, 34 which extends from regions of a person's spine, shown fragmentarily at 36. In this figure, device 20 is being employed as a distraction device to produce a spacing increase between ribs 32, 34, as is indicated generally by oppositely directed arrows 38, 40.

As will be more fully explained also shortly, there are several different ways in which the opposite ends of a device, like device 20, may be connected drivingly to a pair of spaced bones, such as ribs 32, 34. These connections, in character, include a solid, or anchored and rigid, connection, a universal-type connection which allows for angular relative motion at the location of connection, and a linear translational connection. Such a linear translational connection is one which allows a certain amount of normal relative motion between a pair of associated bones, with such motion residing in a range of motion which is associated, as will be explained, with a lessening of what is referred to herein as a currently existing condition of pre-stress in a device such as device 20, notwithstanding the interconnected presence of this device between a pair of such bones. FIGS. 8 and 9 herein illustrate schematically such translational connections, and these will be described fully later herein.

It should be understood that the devices made in accordance with the present invention as illustrated herein, are illustrated only in highly schematic forms, inasmuch as the exact details of their constructions, beyond the presences therein of certain operative components generally, do not form any part of the present invention, and are renderable entirely within the skill level of those generally skilled in the relevant art. Such specific device configurations, and device sizes, as well as device force-applying capabilities, are selectable by a designer/user of the present invention, and they may be tailored to fit specific locations and tasks of bone spacing within a patient's anatomy.

As will be developed below herein, device 20 in FIG. 1, as is also true with respect to the similar, length-change, bone-spacing devices shown in FIGS. 2-5, inclusive, are enabled, through selective RF activation, to change their lengths over time in accordance with the directions of bone-spacing adjustments which are to be made, thus to enable a physician to progress corrective action in dealing with a thoracic definciency syndrome and/or scoliosis issue.

In FIG. 2, device 22 is a contracting rather than a distracting device. Its opposite ends are anchored to a pair of next-adjacent ribs 42, 44 which extend from a patient's spine 36, with device 22 operating to draw these two ribs toward one another, as illustrated generally by arrows 46, 48. In basically all respects except for the fact that device 22 is intended to operate in a contraction mode, this device is fundamentally the same in basic construction as previously mentioned device 20 shown in FIG. 1. The opposite end connections provided for device 22, relative to ribs 42, 44, may take any one of the three forms of connections suggested above in the discussion relating to FIG. 1. Other specific forms of connections, not illustrated herein, may also be used.

In FIG. 3, another form of bone-spacing operation is illustrated wherein a rib 50, extending from spine 36, has had a portion 50a removed to leave spaced rib portions 50b, 50c, between which previously mentioned bone-spacing device, or apparatus, 24 has been drivingly interconnected. Device 24 is essentially the same in construction as previously mentioned device 20. It is installed to operate as a distraction device suitable for applying a separation increasing force, as illustrated generally by arrows 52, 54, between rib portions 50b, 50c.

In FIG. 4, here what is generally and schematically illustrated are both of the conditions of thoracic insufficiency syndrome and scoliosis, regarding which previously mentioned bone-spacing devices 26, 28, 30 have been implanted and appropriately placed to effect spacing-corrective action.

With regard to the schematic illustration of skeletal bones pictured in FIG. 4, included are the spine, here designated 56, five ribs shown at 58, 60, 62, 64, 66, and the pelvis 68 which includes the iliac buttress portion thereof shown at 68a. Device 26 is drivingly interposed ribs 60, 62 to operate in essentially the same manner illustrated for device 20 in FIG. 1. Similarly, bone-spacing device 28 is drivingly interposed ribs 64, 66, also to produce a spacing increase in this case between ribs 64, 66.

Device 30, which is also designed to act as a spacing-increase distraction device, is interconnected drivingly between rib 58 and iliac buttress portion 68a of pelvis 68. Device 30 is so connected through a pair of elongate extenders, such as extenders 70, 72, and a bone screw having a universal-joint-type portion shown generally at 74 in FIG. 4. Device 30 is intended to correct, by straightening, a scoliosis condition in spine 56. It is shown placed to do this by producing a spacing increase between rib 58 and iliac buttress 68a, as is generally indicated by arrows 76, 78 in FIG. 4.

As was mentioned earlier with respect to device 20 in FIG. 1, all of the bone-spacing devices pictured in FIG. 1-4, inclusive, have the characteristic of being RF-activateable to produce progressive spacing-change force application between specific bones to which they are drivingly connected.

It is important to note with respect to the situations illustrated in FIGS. 1-4, inclusive, that the bone-spacing devices shown therein have been implanted for use each in what is referred to herein as a single-surgery procedure. After this procedure, and assuming that there is no reason subsequently to remove these implanted devices, their bone-spacing actions are fully adjustable from outside, and out of contact with, the associated patient through directing RF energy toward them, as will now be more fully explained.

Attention is here directed particularly to FIGS. 5-7, inclusive. In these figures, the basic component make-ups of each of the bone-spacing devices pictured in FIGS. 1-4, inclusive, are illustrated in the context of describing, basically, the working structures of devices 20, 22. It should be recalled at this point that device 20 is a distraction device which is intended to increase the spacing between a pair of associated bones, whereas device 22 is one which operates in a contraction mode to reduce the spacing between a pair of bones. FIG. 5 is employed herein in the description of the invention as an illustration suitable for describing the overall structures of each of devices 20, 22. It is worth recalling also that devices 24, 26, 28, 30 are all essentially the same as device 20 in construction and basic operation.

Referring specifically to FIG. 5, the bone-spacing device 20, 22 therein includes an elongate housing, shown schematically by horizontal lines 80, 82, a pair of spaced, relatively moveable bone drivers 84, 86, an elongate coil spring 88 which is interposed drivers 84, 86, and drivingly connected thereto, as indicated at 90, 92 in FIG. 5, and an elongate mass 94 of an appropriate RF-sensitive constraining material, such as a suitable, RF-relaxable, oriented polymer material, examples of which include various polyethylene and polyacetal materials, which embeds, as can be seen, a substantial length portion of spring 88.

Spring 88 is also referred to herein as a unidirectionally pre-stressed, length-change, spring-force component. In FIG. 5, spring 88 is illustrated in what is referred to herein as a pre-stressed condition which may be either a condition of tension or a condition of compression, depending upon whether the overall bone-spacing device is to operate in compression as a bone-spacing increaser, or in tension as a bone-spacing reducer. In the condition of components illustrated in FIG. 5, spring 88 is restrained against self-biased return to what is referred to herein as a relaxed condition by virtue of the fact that it has a substantial portion of its length embedded in medium 94. A very suitable spring material, and there are many, is a Cobalt-Chromium-Nickel alloy material, such as that made by Alloy Wire International, Ltd. in the U.K. sold under the registered trademarks Phynox® and Elgiloy®.

Medium 94 is also referred to herein as a two-state, RF-activated, stress-control constraining medium which has a hardened state (that in which it is illustrated in FIG. 5) which is changeable to a softened, fluid state under the influence of a field of RF energy having the appropriate characteristics associated with the RF-sensitively of medium 94.

It is significant to note the illustrated, embedded condition of spring 88 with respect to medium 94. One of the important aspects of this “embedded/embedding” relationship between spring 88 and medium 94 will be discussed shortly.

Bone drivers 84, 86 are connected through coupling structures, or connections, shown schematically at 96, 98 to a pair of spaced bones B₁ and B₂, respectively. Connections 96, 98 may take on any one of the earlier-described connections herein.

With the device of FIG. 5 appropriately implanted in a patient between bones B₁ and B₂, medium 94 responds to an appropriate, associated field of RF energy to shift toward and into its softened, fluid state, thus allowing spring 88 to change in length in a direction which tends to minimize the pre-stressed stress level within the spring. The fact that spring 88 is embedded throughout a substantial portion of its length within medium 94 results in a control capability, from the outside of a patient, to make precision and modest progressive changes in the overall effective length of the device illustrated in FIG. 5. In other words, medium 94 is deployed in an interconnection directly with spring 88, whereby a substantial portion of the mass of medium 94 resists, or constrains, length change in spring 88, except under conditions with medium 94 being shifted into its softened, fluid state under the influence of RF energy. It is this nature of interengagement which is featured in the present invention that distinguishes it dramatically from the structures shown in the '746 and '984 patents mentioned above.

If the device pictured in FIG. 5 is intended to be a distraction device to increase the spacing between bones B₁ and B₂, spring 88, as pictured in FIG. 5, is in a pre-stressed condition of compression whereby it tends to self-bias toward a natural relaxed condition by increasing its length.

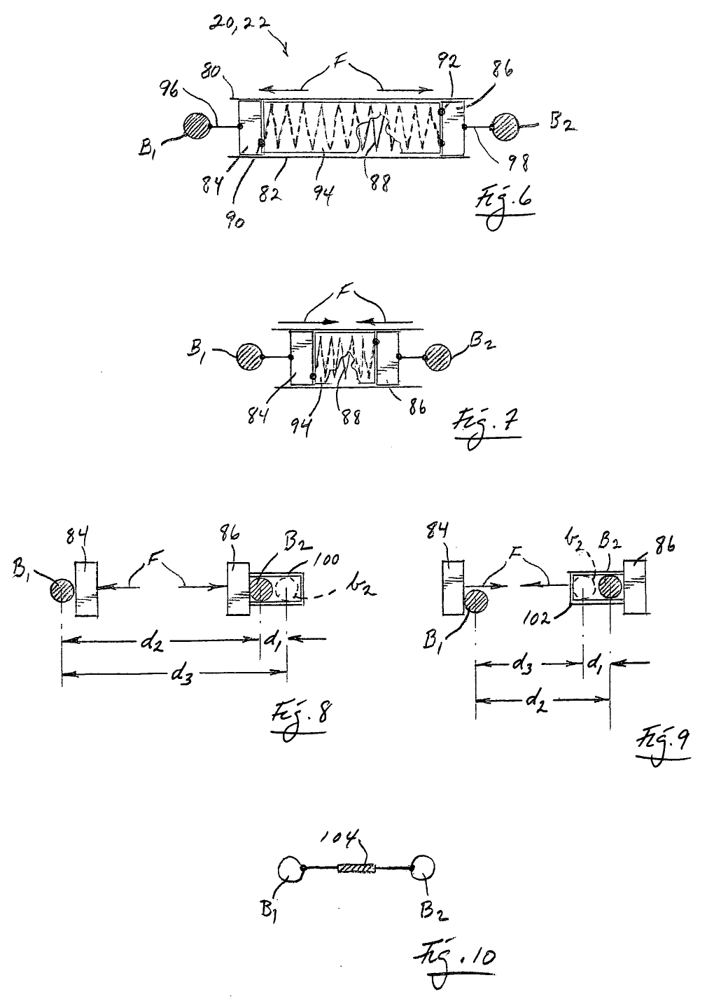

FIG. 6 is employed herein to illustrate distraction behavior by lengthening of spring 88 under circumstances where, in fact, spring 88 is in a pre-stressed condition of compression. Specifically, FIG. 6 illustrates that a force F, which is exerted on bones B₁ and B₂ due to softening of medium 94, preferably in stages, to allow controlled extension of spring 88, has increased the spacings of bones B₁ and B₂, as compared with the spacing for these two bones pictured in FIG. 5.

Similarly, FIG. 7 illustrates a condition wherein the device pictured in FIG. 5 is pre-stressed in a tension condition, whereby it is enabled to decrease the spacing between a pair of bones, such as bones B₁ and B₂.

Turning attention now to FIGS. 8 and 9, these two figures are related to FIGS. 6 and 7, respectively. Thus, FIG. 8 illustrates a distraction, spacing-increase device, and FIG. 9 illustrates a contraction spacing-change device. FIGS. 8 and 9 are included herein specifically to illustrate schematically a type of operative connection which may exist between one or both of bone drivers 84, 86 and a pair of bones, here also labeled B₁ and B₂. In FIGS. 8 and 9, it is only bone driver 86 which is illustrated with a structure which permits such a translational range of normal, spacing-change relative motion between bones B₁ and B₂.

Thus, in FIG. 8, a longitudinally outwardly extending, bone-surrounding structure 100 is formed with, or joined to, bone driver 86 to permit bone B₂, which is nominally drivingly engaged with the right side of driver 86 in FIG. 8, to shift from its solid-outline position in FIG. 8 toward and to its dashed-outline position shown at b₂. This limited range of motion is shown in FIG. 8 at d₁ representing the spacing difference between spacings d₂ and d₃. The range of translational relative motion thus permitted bone B₂ in relation to bone B₁ in a normal-motion manner of behavior, corresponds to the range of motion shown at d₁. This range is associated with a lessening of the existing, current compression condition, represented by F, in spring 88 (which spring is not specifically pictured in FIG. 8). A consequence of this behavior is that for each condition of adjusted length within the bone-spacing device interposed bones B₁ and B₂, a reduction-spacing change is not permitted between these two bones, whereas a limited range of translational normal motion is permitted as indicated by range d₁.

In FIG. 9, a bone-surrounding extension 102 is shown on the inwardly (to the left) facing side of bone driver 86, with, in this circumstance, bone B₂ being permitted to move toward and away from bone B₁, to a dashed-outline position shown at b₂, within the range also indicated at d₁ in FIG. 9.

FIG. 10 illustrates schematically a bone-spacing device 104 which is interposed two bones shown at B₁ and B₂. This figure is provided to illustrate, and to help to visualize, three additional kinds or conditions of bone-spacing utility of the invention. In each of these three conditions, bone B₁ represents a vertebral body. In one of these conditions, bone B₂ represents a next-adjacent vertebral body. In another one of these conditions, bone B₂ represent a rib. In the third one of these conditions, bone B₂ represents the pelvis.

Different versions of a bone-spacing device, or apparatus, made in accordance with the present invention have thus been described and illustrated herein. In each embodiment of such a device, in accordance with practice of the invention, the elongate biasing spring component, 88, has a substantial portion of its length embedded in the mass 94 of constraining material which, in the absence of an appropriate field of RF energy, is in a hardened state. Relaxation of this state to a softened, fluid state, through irradiating medium 94 with appropriate RF energy, allows spring 88 to change in length so as to impart driving forces to bone drivers 84, 86, progressively to change the spacing between bones B₁ and B₂ in the desired direction. As was mentioned earlier herein, the fact that spring 88 is embedded along a long length portion within medium 94 results in a reality of enabling a user of the invention to implement high precision control over progressive force-application bone-spacing changes.

It is entirely possible that, for example, in a situation like that illustrated in FIG. 4, each of the three bone-spacing devices illustrated therein might be constructed with specifically different RF-activated constraining medium structures which respond, for example, to different frequencies of RF energy. With such a situation existing, it will be clear that it is possible for a practitioner of the invention to enable a length change to take place selectively in any one, but not all simultaneously, of the three devices shown in FIG. 4.

With regard to practice of the present invention, there are several ways to think about and characterize the bone-spacing methodology offered by the present invention. One way is to express this methodology as being a single-surgery method for producing a change in the spacing existing between a pair of selected, spaced bones in a patient, including the steps of (a) implanting, in a condition of driving interposition selected bones, elongate bone-spacing apparatus including a constraining medium which can change between hardened and softened states in response to associated RF energy, and (b) from outside the subject patient, selectively communicating to the constraining medium in the implanted bone-spacing apparatus RF energy of the character associated with the particular constraining medium in that apparatus.

Another way of expressing this methodology is to describe it as being a method for producing a change in the spacing existing between a pair of selected, spaced skeletal bones in a patient, including the steps of (a) implanting an elongate, unidirectional, distributed-RF-controllable, length-change force-applying instrumentality in an operative driving condition between a pair of such bones, (b) selectively subjecting the implanted instrumentality to RF activation distributed along its length in a manner wherein length-change action in the instrumentality occurs therealong at plural distributed points of RF control (i.e., the various points of interengagement between a biasing spring and a constraining medium, and (c) as a consequence of the subjecting step, effecting a controlled spacing change between the selected bones.

The methodology of the invention also includes the step of enabling a selected character of relative spacing-change motion to take place between relevant bones, notwithstanding the presence therebetween of an implanted spacing-change instrumentally.

One can thus see that the device and apparatus of the present invention is relatively simple and inexpensive in construction and lends itself to manufacture in many different sizes, configurations and force-applying levels.

It can be used easily, and importantly in what is a single-surgery, single-invasion practice, and it enables controlled, progressive, force-application correction in the spacings existing between different bones which are relevant to the thoracic insufficiency syndrome and scoliosis deformity conditions described earlier herein. It enables this behavior, moreover, in a highly controllable precision manner.

Precision, control over length change in the device of the invention—clearly attainable by this device—appears to derive from the fact of long-length embedment of a portion of the device's biasing spring in the phase-change, RF-sensitive constraining medium. A substantial mass of this medium engages the spring along a large portion of the spring's body, and with softening of the medium, the spring must therefore work with substantial effort at many length-distributed regions to overcome the anti-spring-relaxation restraining effect of the medium. This action is sharply distinguishable from the earlier-mentioned prior art action, wherein but a very small surface interengagement exists between a bone driver and a constraining medium, resulting in a relatively modest task for a constrained, bone driver, namely, to move only a relatively tiny mass of softened constraining material in order to enable shifting toward relaxation of an associated biasing spring. “Tiny mass” interengagement, as distinguished from “substantial mass” interengagement as proposed by the present invention, can result in inadvertent over-adjustment in a force-application spacing change, and thus in less precision control over the desired staging of bone-position adjustments. Precision adjustments thus become a difficult challenge under such a prior art structural and behavior situation.

The structure an operative methodology of the present invention have thus been described and illustrated in several preferred embodiments and manners of practice. Those generally skilled in the relevant art will appreciate that variations and modifications may be made without departing from the spirit of the invention, and it is intended that all such variations and modifications will come appropriately within the scopes of the several claims to the invention which now follow.

I claim:

1. Single-surgery, elongate selectively adjustable, length-change bone-spacing apparatus comprising
an elongate, unidirectionally pre-stressed, length-change, spring-force component having a relaxed condition toward which it is self-biased, and in which relaxed condition it is either one of longer or shorter than when it is in its pre-stressed condition,
a pair of spaced, relatively moveable bone drivers coupleable selectively to selected, spaced bones, and drivingly connected, one each, to opposite ends of said spring-force component, and
a two-state, RF-activated, stress-control constraining medium embedding at least a portion of the length of said spring-force component, and having a hardened state wherein it immobilizes said length portion against any length change, and a softened, fluid state to which it converts reversibly under the influence of received radio-frequency (RF) energy of a defined character which is specific to said constraining medium, and in which softened state, the constraining medium permits length change of said length portion.

2. The bone-spacing apparatus of claim 1, wherein said spring-force component takes the form of an elongate coil spring.

3. Single-surgery, elongate, selectively adjustable, length-change bone-spacing apparatus comprising
an elongate, unidirectionally pre-stressed, length-change, spring-force component having a relaxed condition toward which it is self-biased, and in which relaxed condition it is either one of longer or shorter than when it is in its pre-stressed condition,
a pair of spaced, relatively moveable bone drivers coupleable selectively to selected, spaced bones, and drivingly connected, one each, to opposite ends of said spring-force component,
a two-state, RF-activated, stress-control constraining medium operatively linked effectively to said spring-force component, and having a hardened state wherein it effectively immobilizes the spring-force component against any length change, and a softened, fluid state to which it converts reversibly under the influence of received radio-frequency (RF) energy of a defined character which is specific to said constraining medium, and in which softened state the constraining medium permits a spring-force-component length change, and
structure for coupling said drivers to a pair of selected, spaced bones in a manner which, when such coupling exits, permits, with respect to any current condition of internal stress then existing in said spring-force component, reversible relative motion between said drivers in a range of motion which is associated with a lessening of the current condition of internal stress in said spring-force component.

4. The bone spacing apparatus of claim 3, wherein said constraining medium embeds at least a portion of the length of said spring-force component.

5. The bone-spacing apparatus of claim 3, wherein said spring-force component takes the form of a coil spring.

6. A single-surgery method for producing a change in the spacing existing between a pair of selected, spaced bones in a patient comprising
implanting, in a position of driving interposition such selected bones, elongate bone-spacing apparatus including a constraining medium as set forth in claim 1, and
from outside the patient, selectively communicating to the constraining medium in the implanted bone-spacing apparatus radio-frequency (RF) energy of the character associated with the constraining medium in that apparatus.

7. The method of claim 6, wherein the selected bones include a pair of next-adjacent ribs.

8. The method of claim 6, wherein the selected bones include split portions of a single rib.

9. The method of claim 6, wherein the selected bones include the pelvis and a rib.

10. The method of claim 6, wherein the selected bones include a first vertebral body, and one of (a) another, next-adjacent vertebral body, (b) the pelvis, and (c) a rib.

11. The method of claim 6 which is practiced in relation to plural pairs of selected bones, including any combination of (a) next-adjacent ribs, (b) adjacent rib portions of a single rib, (c) the pelvis and one or more rib(s), (d) two next-adjacent vertebral bodies, (e) a vertebral body and a rib, and (f) a vertebral body and the pelvis.

12. A single-surgery method for producing a change in the spacing existing between a pair of selected, spaced bones in a patient comprising
implanting, in a position of driving interposition such selected bones, elongate bone-spacing apparatus including a constraining medium as set forth in claim 3, and
from outside the patient, selectively communicating to the constraining medium in the implanted bone-spacing apparatus radio-frequency (RF) energy of the character associated with the constraining medium in that apparatus.

13. The method of claim 12, wherein the selected bones include a pair of next-adjacent ribs.

14. The method of claim 12, wherein the selected bones include split portions of a single rib.

15. The method of claim 12, wherein the selected bones include the pelvis and a rib.

16. The method of claim 12, wherein the selected bones include a first vertebral body, and one of (a) another, next-adjacent vertebral body, (b) the pelvis, and (c) a rib.

17. The method of claim 12 which is practiced in relation to plural pairs of selected, spaced bones, including any combination of (a) next-adjacent ribs, (b) adjacent rib portions of a single rib, (c) the pelvis and one or more rib(s), (d) two next-adjacent vertebral bodies, (e) a vertebral body and a rib, and (f) a vertebral body and the pelvis.

18. A method for producing a change in the spacing existing between a pair of selected, spaced skeletal bones in a patient comprising
implanting an elongate, unidirectional, distributed-RF-controllable, length-change force-applying instrumentality in an operative driving condition between a pair of such bones,
selectively subjecting the implanted instrumentality to RF activation distributed along its length in a manner wherein length-change action in the instrumentality occurs therealong at plural, distributed points of RF control, and
as a consequence of said subjecting, effecting a controlled spacing change between the selected bones.

19. The method of claim 18 which further comprises enabling a selected character of relative spacing-change motion between the selected bones notwithstanding the presence therebetween of the implanted instrumentality.
